(19) United States
(12) Patent Application Publication
(10) Pub. No.: US 2003/0069447 A1
(43) Pub. Date: Apr. 10, 2003

(54) Acetal hydroxylamine compounds

(76) Inventors: Paul Howard Briner, Cowley (GB); Andrew Flood, Oxford (GB)

Correspondence Address: BANNER & WITCOFF, 1001 G STREET N W, SUITE 1100, WASHINGTON, DC, 20001, US

(21) Appl. No.: 10/182,735

(22) PCT Filed: Feb. 16, 2001
(86) PCT No.: PCT/GB01/00668

(30) Foreign Application Priority Data
Feb. 16, 2000 (GB) 000347.9

(51) Int. Cl. C07C 239/10

(52) U.S. Cl. 564/301

(57) ABSTRACT

Compounds of formula (I): R₁CH₂(OR₂)ONH₂, wherein R₁ is hydrogen or methyl and R₂ is a C₁-C₆ alkyl group, or a salt thereof are useful as reagents for the synthesis of biologically active hydroxyaminocarbonyl- and N-formyl-N-hydroxylamino-containing compounds.

The present invention relates to acetal hydroxylamine compounds, useful as reagents for the synthesis of hydroxyaminocarbonyl- and N-formyl-N-hydroxylamino-containing compounds.

### BACKGROUND TO THE INVENTION

Certain hydroxamic acid derivatives possess useful biological activities Examples include compounds that inhibit urease (Odake S et al., Chem. Pharm. Bull., 1992, 40, 2764-2768), trypanosome glycerol-3-phosphate oxidase (Grady et al., Mol. Biochem. Parasitol., 1986, 19, 231-240), dehydropeptidase-1 (EP-B-276,947), ribonucleotide reductase (Farr R A et al., J. Med. Chem., 1989, 32, 1879-1885), 5-lipoxygenase (Kerdesky F A J et al., Tetrahedron Lett., 1985, 26, 2134-2146; U.S. Pat. No.-4,731,382), substance P degradation (Laufer R et al., Eur. J. Biochem., 1985, 150, 135-140), cardiovascular metalloproteinase enzymes (Turbanti L et al., J. Med. Chem., 1993, 36, 699-707; WO-9428012) and matrix metalloproteinase enzymes (Schwartz M A, Van Wart H E, Prog. Med. Chem., 1992, 29, 271-334).

Certain N-formyl-N-hydroxylamino-containing compounds also possess useful biological activities. Examples include compounds which inhibit enkephalinase (eg U.S. Pat. No. 4,738,803), angiotensin converting enzyme (eg WO 97/38705), matrix metalloproteinase enzymes (eg EP-B-0236872 and WO 95/33709) and bacterial peptide deformylase (WO 99/39704).

Preparation of hydroxamic acid derivatives has generally required the conversion of a carboxyl group of a precursor compound to a hydroxyaminocarbonyl group by reaction of the carboxyl group or an activated derivative thereof with hydroxylamine or an N—, O—, or N,O-protected hydroxylamine. The most common hydroxylamine reagents for this purpose have been hydroxylamine itself, and O-benzylhydroxylamine. The former suffers from lack of N,O-selectivity, leading to unwanted byproducts The latter requires hydrogenolysis for removal of the benzyl protecting group, with consequent risk of precious metal contamination of the product.

Preparation of N-formyl-N-hydroxylamino-containing compounds has also generally, involved the introduction of a hydroxyamino group into a precursor compound using hydroxylamine or N—, O— or N,O-protected hydroxylamine, followed in this case by formylation of the nitrogen. The same disadvantages are inherent is these processes as in the preparation of hydroxamic acid derivatives.

There is therefore a need in the art for reagents for the generation of a hydroxyaminocarbonyl group from a carboxyl group or for the introduction of a hydroxyamino group, as part of the synthesis of actual and potential pharmaceuticals.

### BRIEF DESCRIPTION OF THE INVENTION

The invention is based on the finding that a novel class of O-protected hydroxylamines, easily accessible by simple chemistry from inexpensive commercially available starting materials, may be used in the synthesis of hydroxyaminocarbonyl- and N-formyl-N-hydroxylamino-containing compounds of the kind sought in the art as actual and potential pharmaceuticals.

### DETAILED DESCRIPTION OF THE INVENTION

According to the present invention there is provided a compound of formula (I)

R₁CH₂CH(OR₂)ONH₂ (I)

wherein R₁ is hydrogen or methyl and R₂ is a C₁-C₆ alkyl group, or a salt thereof.

The term “C₁-C₆ alkyl” means a straight chain or branched alkyl group of from 1 to 6 carbon atoms inclusive. Thus, in the compounds (I), R₂ may be, for example, methyl, ethyl, n- or iso-propyl, n-, iso-, sec- or tert-butyl, n-pentyl or n-hexyl.

Presently, it is preferred that R₁ be hydrogen, and R₂ be n- or iso-butyl.

Salts of the reagents of the invention may be those formed with weak organic acids, such as oxalic, citric acid or tartaric acid.

Compounds of the invention are useful in a process for converting a carboxyl group —COOH present in a precursor compound to a hydroxamic acid group —CONHOH, which process comprises condensation of the said carboxyl acid group with the amino group of a compound of formula (I), whereby the said carboxyl group is converted to a group of formula (II).

—CONHOCH(OR₂)(CH₂R₁) (II)

and subsequently the group (II) is converted to a hydroxamic acid group by acid hydrolysis.

The carboxyl group of the precursor compound is preferably activated for the condensation as an activated ester, eg the 1-hydroxybenzotriazole ester, an acid halide, eg the chloride, or an acid anhydride. Acid hydrolysis may be effected under mild conditions, for example using hydrochloric acid at ambient temperatures, for example about 20° C.

Compounds of the invention are also useful in a process for converting a moiety of formula (IIIA), (IIIB), (IIIA¹) or (IIIB¹) present in a precursor compound

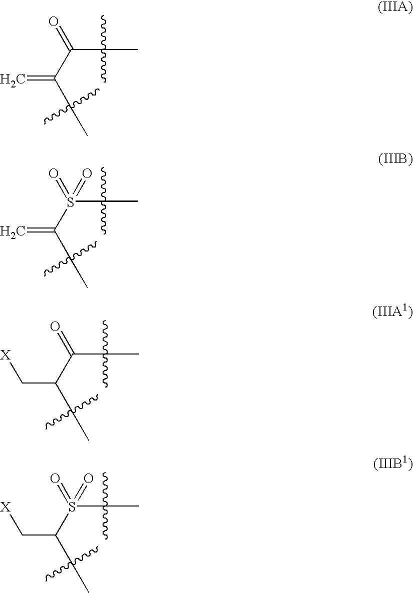

wherein X is a leaving group, for example a triflate group, to an N-formylhydroxylamino divalent moiety of formula (IVA) or (IVB)

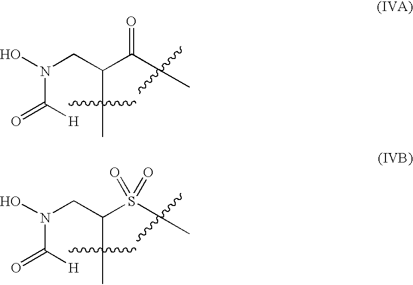

which process comprises reacting the said precursor compound with a compound of formula (I) as claimed in any of claims 1 to 5 to convert moiety (IIIA) or (IIIA¹) to a moiety (IIIC). or to convert moiety (IIIB) or (IIIB¹) to a moiety (IIID)

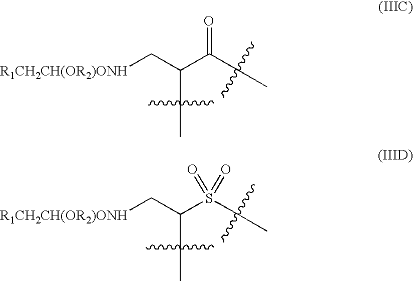

then converting moiety (IIIC) or (IIID) to moiety (IIIE) or (IIIF) by acid hydrolysis

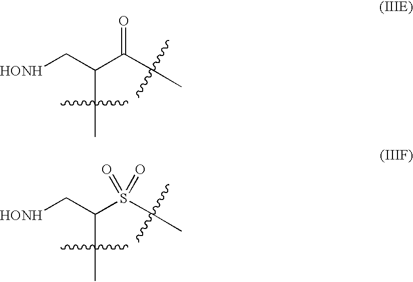

and, at the same time or subsequently, N-formylating the nitrogen of (IIIE) or (IIIF) to form moiety (IVA) or (VB).

Again, acid hydrolysis may be effected under mild conditions, for example using hydrochloric acid at ambient temperatures, for example about 20° C. N-formylation of (IIIA) may be effected using formic acetic anhydride, or 1-formylbenzotriazole.

Compounds (I) of the invention are also useful reagents in the synthesis of compounds containing an N-substituted β-lactam moiety of formula (V)

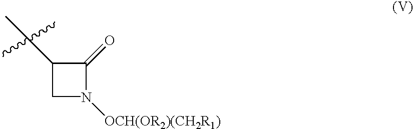

by reaction of a compound containing a 1-carboxy-2-hydroxy-1-yl moiety of formula (VA)

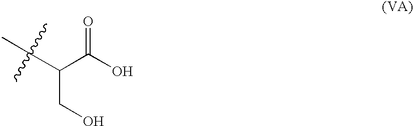

with a compound of the invention (I) thereby converting moiety (VA) to a moiety (VB)

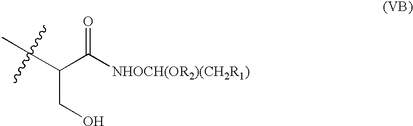

then cyclising moiety (VB) by internal amino-hydroxyl condensation to form the desired N-substituted β-lactam compound (V).

N-substituted β-lactam compounds (V) may be ring opened by acid hydrolysis to yield compounds containing moieties of formula (VI)

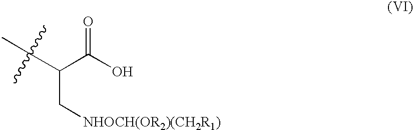

which may then be N-formylated to form compounds containing moieties of formula (VIA)

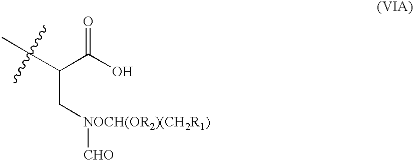

which may in turn be acid hydrolysed to N-formyl-N-hydroxylamino moieties (VB)

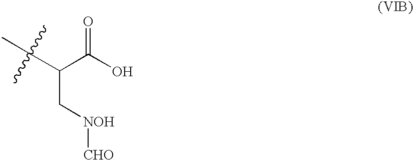

Compounds of formula (I) may be prepared by acid catalysed reaction of a vinyl ether of formula (VII)

R₁CH═CHOR₂ (VII)

with an N,N-diprotected hydroxylamine, followed by removal of the nitrogen protecting groups. For example, the diprotected hydroxylamine may be an N-hydroxy succinimide or analogue such as N-hydroxyphthalimide, which may be reacted with (V) in the presence of pyridinium tosylate, p-toluenesulphonic or trifluoroacetic acid or acidic resin as catalyst, and the resultant imide nitrogen converted to a free amino group by base hydrolysis, for example using propylamine.

The following Examples illustrate the invention and its utility. Example 1 describes the synthesis of specific compounds of the invention. Example 2 describes the use of compounds of the invention for the conversion of a carboxylic acid model compound to the corresponding hydroxyaminocarbonyl analogue. Example 3 describes the use of a compound of the invention in the synthesis of a known hydroxamate matrix metalloproteinase inhibitor. Example 4 describes the use of a compound of the invention in the synthesis of an N-formyl-N-hydroxylamino compound having antibacterial properties.

The following abbreviations have been used in the Examples:

| | |
|---|---|
| EtOAc | Ethyl acetate |
| PPTS | Pyridinium tosylate |
| TFA | Trifluoroacetic acid |
| HOBt | 1-Hydroxybenzotriazole |
| DCC | Dicyclohexylcarbodiimide |
| DCU | Dicyclohexylurea |
| THF | Tetrahydrofuran |
| MTBE | Methyl-t-butyl ether |

### EXAMPLE 1

### Synthesis of Hydroxylamino-Acetals

A range of hydroxylamino-acetals (3) were made using the synthesis outlined below (Scheme 1). Small amounts of by-products, oxime (4) and acetal (5), are produced in the reaction, but the recovered hydroxylamino acetal (3) may be purified if desired.

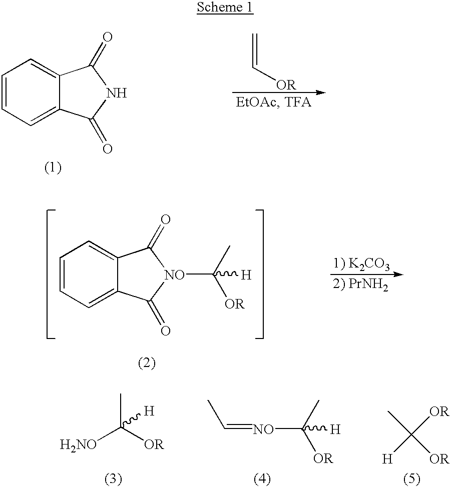

O-(1-Butoxy-ethyl)-hydroxylamine (3, R=nBu)

To a suspension of N-hydroxyphthalimide (50 g, 0.31 mol.) in EtOAc (125 ml.) was added PPTS (7.70 g, 0.03 mol) followed by n-butyl vinyl ether (61.35 g, 0.61 mol) over about 5 minutes at room temperature. After stirring for about 3 hours, the reaction mixture cleared and tic (40% EtOAc/hexane) indicated that the O-protection was complete. The reaction mixture was stirred out for 30 mins with K₂CO₃ (5.14 g, 0.037 mol. 0.12 eq) in order to neutralise the PPTS. N-Propylamine (36.2 g, 0.61 mol) was added slowly to the cooled reaction mixture while maintaining the temperature below 20° C. After about 3.5 hours, a thick white precipitate of the bis(monopropyl)phthalamide formed and the mixture was allowed to stir out for 16 hours to complete the N-deprotection. Diethylphthalate (50 ml) was added to the reaction mixture as a distillation ‘heel’ and the EtOAc and propylamine were removed by distillation at atmospheric pressure. The temperature was allowed to drop to 30° C. and the product distilled at reduced pressure (40-50° C. @ 5 Torr). Yield of product=37.6 g. ¹HNMR showed product and two impurities; oxime (4), 9 mol % and acetal (5), 6 mol %.

250 MHz ¹HNMR (CDCl₃,TMS): δ=0.94 (t, J=7 Hz, 3H, CH₂CH₃), 1.31 (d, J=6 Hz, CH₃), 1.34-1.65 (m, 4H, CH₂CH₂), 3.44-3.77 (m, 2H, OCH₂), 4.78 (q, J=6 Hz, CH), 5.27 (bs, 2H, NH₂).

O-(1-Propoxy-ethyl)-hydroxylamine (3, R=nPr)

Prepared as O-(1-butoxy-ethyl)-hydroxylamine.

250 MHz ¹HNMR (CDCl₃,TMS): δ=0.93 (t, J=7 Hz, 3H, CH₂CH₃), 1.32 (d, J=6 Hz, 3H, CH₃), 1.63 (m, 2H, CH₂CH₃), 3.42-3.71 (m, 2H, OCH₂), 4.78 (q, J=6 Hz, 1H, CH), 5.27 (bs, 2H, NH₂).

O-(1-Ethoxy-ethyl)-hydroxylamine (3, R=Et)

Prepared as O-(1-butoxy-ethyl)-hydroxylamine

250 MHz ¹HNMR (CDCl₃,TMS): δ=1.24 (t, J=7 Hz, 3H, CH₂CH₃), 1.32 (d, J=6 Hz, 3H, CH₃), 3.51-3.84 (m, 2H, OCH₂), 4.79 (q, J=6 Hz, 1H, CH), 5.28 (bs, 2H, NH₂).

O-(1-Isobutoxy-ethyl)-hydroxylamine (3, R=iBu)

To a suspension of N-hydroxyphthalimide (2.445 kg, 15.0 mol.) in EtOAc (12.22 l) was added TFA (120 ml, 1.5 mol) followed by iso-butyl vinyl ether (1.65 kg, 16.5 mol) over about 15 mins at room temperature. After stirring out for 21 hours, the reaction mixture was clear and tic (40% EtOAc/hexane) indicated that the O-protection was complete. The reaction mixture was stirred out with K₂CO₃ (621 g, 4.5 mol. 0.3 eq) for 90 mins, filtered (Nutsche) and the stir-out repeated with more K₂CO₃ (414 g, 0.2 eq) for 60 mins. After filtration, n-propylamine (1.77 kg, 30 mol) was added slowly to the cooled reaction mixture (ice/water bath) while maintaining the temperature below 20° C. and allowed to stir out for 16 hours. The precipitated bis(monopropyl)phthalamide was filtered off, rinsed through with EtOAc (2×500 ml) and the combined organics were treated with diethylphthalate (2.5 l) as a distillation ‘heel’. The EtOAc and propylamine were removed by distillation at atmospheric pressure, the temperature allowed to drop to 30° C. and the product distilled at reduced pressure (62-77° C. @ 25−12 Torr). Yield of product (corrected)=1281 g, 64%. ¹HNMR showed product and two impurities; oxime (4), 6 mol % and acetal (5), 3 mol %

250 MHz ¹HNMR (CDCl₃,TMS): δ=0.93 (d, J=7 Hz, 6H, CMe₂), 1.31 (d, J=5 Hz, 3H, CH₃), 1.86 (m, 1H, CHMe₂), 3.24-3.57 (m, 2H, OCH₂), 4.76 (q, J=5 Hz, 1H, CH), 5.28 (bs, 2H, NH₂).

### EXAMPLE 2

### Synthesis of Hydroxamic Acids Using Hydroxylamino-Acetals.

Using the synthesis outlined in Scheme 2, N-hydroxy-2-phenyl-acetamide (8) was prepared in two steps via the hydroxamates (2a-2d).

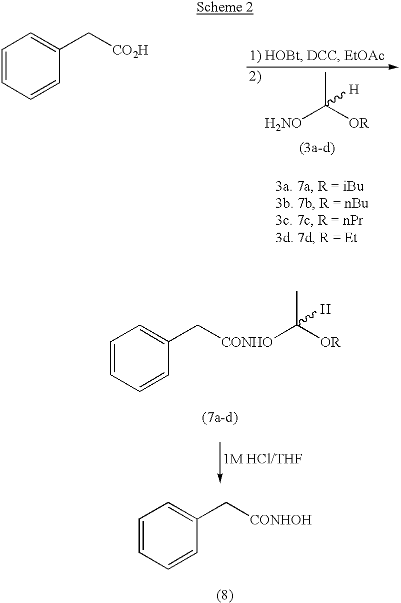

Step 1

A solution of phenylacetic acid (5.0 g, 37 mmol) in EtOAc (50 ml) was treated with HOBt hydrate (37 mmol, 5.65 g corrected for water content) at ambient temperature. To this mixture was added DCC (1.89 g, 37 mmol) in EtOAc (20 ml) over 15 mins on an ice-water bath. When the addition was complete. the bath was removed and the reaction mixture was stirred out for 2 hours at ambient temperature. The reaction mixture was cooled to 0-5° C. and filtered to remove the DCU. The filter cake was washed with cold EtOAc (10 ml) and the combined filtrates assumed to contain 37 mmol of the HOBt ester of phenylacetic acid.

The above filtrates were treated with O-(1-isobutoxy-ethyl)-hydroxylamine, (3a, 37 mmol, 5.53 g) over 5 mins at ambient temperature and then stirred out for 3 hours. The reaction mixture was washed with 2M Na₂CO₃ (2×10 ml), saturated brine (1×5 ml), dried over MgSO₄ and freed of solvent to give the protected hydroxamate (7a), which was characterised by ¹HNMR.

250 MHz ¹HNMR (CDCl₃,TMS): δ=0.81 (d, J=6 Hz, 6H, CMe₂), 1.34 (d, J=6 Hz, 3H, CH₃), 1.75 (m, 1H, CHMe₂), 3.16-3.47 (m, 2H, OCH₂), 3.58 (bs, 2H, PhCH₂), 4.90 (bs, 1H, CH), 7.31 (m, 5H, Ph), 7.80 (bs, 1H, NH).

Step 2

A 9 mmol aliquot of the above hydroxamate (7a) was dissolved in THF (30 ml) and treated with 1M HCl (5 ml). The mixture was stirred for 16 hours at ambient temperature then rotary evaporated to dryness. The residue was dissolved in EtOAc (100 ml) washed with 18 wt % brine (2 ml), D.I. water (2 ml) and dried (MgSO₄). Rotary evaporation of the solution gave the hydroxamic acid (1.26 g, 90%); the product was characterised by ¹HNMR.

250 MHz ¹HNMR (d₆-DMSO,TMS): δ=3.56 (s, 2H, CH₂), 7.28 (m, 5H, Ph), 10.63, 12.27 (CONHOH).

The same methods were used to synthesise N-hydroxy-2-phenyl-acetamide (8) using the range of hydroxylamino-acetals shown in the following Table:

| Hydroxylamino-acetal | R | % yield of (8) |
|---|---|---|
| O-(1-butoxy-ethyl)- | n-Bu | 85 |
| hydroxylamine (1b) | | |
| O-(1-propoxy-ethyl)- | n-Pr | 92 |
| hydroxylamine (1c) | | |
| O-(1-ethoxy-ethyl)- | Et | 67 |
| hydroxylamine (1d) | | |

### EXAMPLE 3

### Synthesis of N¹-[2,2-Dimethyl-1S-(pyridin-2-ylcarbamoyl)-propyl]-N⁴-hydroxy-2R-isobutyl-3S-methoxy-succinamide

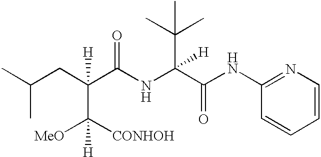

The title compound, which is a known matrix metalloproteinase inhibitor (see WO 99/25693), was prepared using the n-butyl hydroxylamino-acetal (3b) according to the synthesis outlined in Scheme 3:

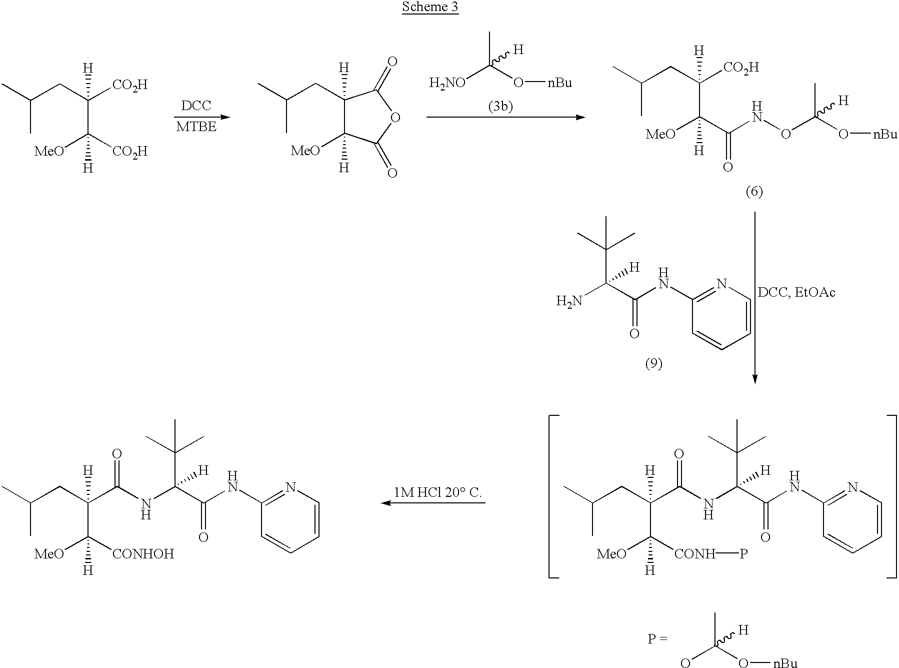

A solution of 3-(R)-carboxyl-2-(S)-methoxy-5-methylhexanoic acid (190 g, 0.93 mol) in MTBE (1000 ml) was cooled to below 5° C. and treated with a solution of DCC (191.3 g, 0.93 mol) in MTBE (240 ml) dropwise over 20 minutes while maintaining the temperature below 15° C. The reaction mixture was allowed to warm to room temperature, stirred out for 1 hour, cooled to below 5° C. and the DCU filtered off. The DCU cake was washed with MTBE (500 ml) and the filtrates combined. The n-butyl hydroxylamino-acetal (3b, 123.8 g contained, 0.93 mol) was added to the filtrates over 5 minutes and stirred out for 16 hours at room temperature. TIc (40% EtOAc/hexane) indicated complete reaction and the ¹HNMR showed the hydroxamate (6) product along with the unreacted oxime (4) and acetal (5). The solvent was removed by rotary evaporation and used in the next stage without further purification.

For the purpose of calculating reagent quantities, the conversion of 3-(R)-carboxyl-2-(S)-methoxy-5-methylhexanoic acid to the hydroxamate (6) was assumed to be quantitative but corrected for the impurities. Solid (S)-2-amino-3,3-dimethyl-N-pyridin-2-yl-butyramide (9, 183.1 g, 0.88 mol) was added to a solution of the hydroxamate prepared above in EtOAc (950 ml). The reaction mixture was cooled to 15° C. and DCC (182.1 g, 0.88 mol) in EtOAc (220 ml) was added over 40 minutes while maintaining the temperature below 20° C. Cooling was applied in the subsequent stir-out to control the exotherm (T<30° C.). It was necessary to add more solvent (800 ml) 10 minutes post-addition in order to keep the reaction mixture mobile as DCU precipitated. The reaction mixture was stirred out for 16 hours at room temperature then extracted with 1M HCl (990 ml) over 30 mins. The DCU was filtered off, the aqueous layer separated and the organic layer extracted for 15 mins with 1M HCl (990 ml) which had first been used to wash through the DCU cake. The organic layer was extracted a third time (300 ml). The combined aqueous layers were covered with EtOAc (3380 ml), stirred vigorously and the pH brought to 8-8.5 with 2M NaOH. The separated aqueous layer was re-extracted with EtOAc (1100 ml). The combined organic extracts were warmed to 50° C. to dissolve crystallised product, washed with saturated brine (400 ml) then D.I. water (400 ml). The mixture was azeotroped dry and the solvent distilled to a product solution volume of 1400 ml (equivalent to approx. 5 ml/g of hydroxamate, 6). The product was allowed to cool and crystallise out, filtered, washed with hexanes (2×300 ml) and dried under reduced pressure at 80° C. Yield=171 g, 45%. HPLC=94.3 area %. ¹HNMR was consistent with the title compound.

250 MHz ¹HNMR (d₆-DMSO, TMS): δ=0.73, 0.82 (2×d, 6H, J=6 Hz, CMe₂), 1.00 (s, 9H, ^{t}Bu), 1.40 (m, 3H, CH₂CHMe₂), 2.92 (m, 1H, H_{b}), 3.12 (s, 3H, OCH₃), 3.48 (d, J=10 Hz, 1H, Hₐ), 4.58 (d, J=9 Hz, 1H, CH ^{t}Bu), 7.10 (dd, J=7, 5.5 Hz, 1H, H_{d}), 7.77 (ddd, J=8, 7, 1 Hz, 1H, Hₑ), 7.94 (d, J=9 Hz, 1H, CONH), 8.05 (d, J=8 Hz, 1H, H_{f}), 8.31 (dd, J=5.5, 1 Hz, 1H, H_{c}), 9.03 (bs, 1H, CONHpyr), 10.25 (bs. 1H, CONHOH), 10.80 (bs, 1H, CONHOH).

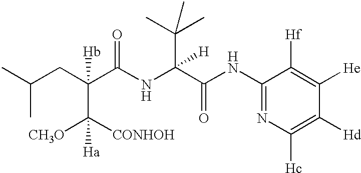

### EXAMPLE 4

### Synthesis of N-[2-(4-Benzyl-piperidin-1-yl)-1-(3 cyano-benzyl)-2-oxo-ethyl]-2-cyclopentylmethyl-3-(formyl-hydroxy-amino )-propionamide.

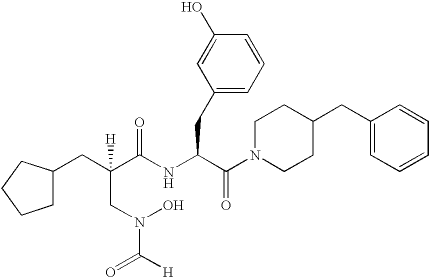

Synthesis of the above compound will be described by reference to reaction schemes 4-6 below.

Step A: Preparation of Acylated Chiral Auxiliary (8) (See Scheme 4)

A solution of (S)-(−)-4-Benzyl-2-oxazolidinone (7, 266 g 1.50 mol) in THF (2500 ml) was cooled to below −70° C. under argon and then a solution of butyllithium (1020 ml, 1.62 mol) added via cannula over 1 hour 20 minutes, maintaining the temperature below −60° C. The reaction mixture was stirred for a further 15 minutes post-addition and then a solution of 3-cyclopentylpropionyl chloride (6, 253 ml, 1.62 mol) in THF (500 ml) was added via cannula under argon over 35 minutes while maintaining the reaction temperature below −60° C.

The reaction mixture was allowed to reach room temperature, quenched with 20% w/v ammonium chloride solution (220 ml) and stirred out for 16 hours. After filtration of the reaction mixture to remove inorganics, THF was distilled off under reduced pressure and replaced with EtOAc (1000 ml). The organic layer was washed with 1M Na₂CO₃ solution (2×500 ml) and then with saturated brine solution (2×300 ml). Hexanes (200 ml) were added to the reaction mixture which was dried by azeotropic distillation of the water. Concentration of the mixture under reduced pressure yielded the product as an off-white solid (8, 462 g,>100%).

¹H NMR showed the presence of small amounts of residual 3-cyclopentylpropionic acid.

¹Hδ(CDCl₃)ppm: 1.49 (11H, m); 2.78 (1H, dd, J=9.4 Hz, 13.4 Hz) 3.30 (1H, dd, 3.3 Hz, 13.5 Hz); 2.94 (2H, m); 4.19 (2H, m); 4.67 (1H, m); 7.27 (5H, m).

Step B: Preparation of Hydroxymethyl Compound (9) (See scheme 4)

A solution of the acylated Evans Auxiliary (8, 14.29 g, 47.4 mmol) in anhydrous DCM (150 ml) was cooled to 0° C. under argon in a dried flask. To this solution was added a solution of 1.0 M TiCl₄ in DCM (50 ml, 50 mmol) followed 10 minutes later by diisopropylethylamine (DIPEA, 91.4 ml, 52.5 mmol). The deep red reaction mixture was stirred out for 1 hour at 0° C. Into another dried, inerted flask, a solution of s-trioxane (5.13 g, 56.9 mmol) in anhydrous DCM (75 ml) at 0° C. was added 1.0M TiCl₄ solution (50 ml, 50 mmol) to give an off-white suspension which was stirred out for 15 minutes. The s-trioxane/TiCl₄ suspension was added via cannular to the starting material mixture at 0° C. for 4 hours then allowed to stir out for 16 hours at ambient temperature.

The reaction mixture was cooled to 0° C., quenched with saturated NH₄Cl solution (200 ml) and the aqueous layer separated and then extracted with DCM (75 ml) The organic layers were combined, washed with 1.0M Na₂CO₃ (150 ml), brine (100 ml) and dried (MgSO₄). The extracts were concentrated under reduced pressure and the residual solid was recrystallised from EtOAc to yield the product as a white solid in two crops (9, 11.10 g, 33.5 mmol).

¹Hδ(CDCl₃)ppm: 1.00-1.25 (2H, m); 1.40-1.90 (9H, m,); 2.25 (1H, dd, J=8.3 Hz, 4.5 Hz); 2.81 (1H, dd, J=9.4 Hz, 13.4 Hz); 3.30 (1H, dd, J=3.3 Hz, 13.5 Hz); 3.85 (2H, m); 4.05 (1H, m); 4.20 (2H, m); 4,70 (1H, m); 7.30 (5H, m).

Step C: Preparation of Carboxylic Acid (10) (See Scheme 4)

A solution of 9 (11.10 g, 33.5 mmol) in THF (200 ml) and deionised water (40 ml) was cooled to 0° C. and treated with 27% aqueous hydrogen peroxide solution (17 ml, 134 mmol) followed by LiOH monohydrate (3.09 g, 73.7 mmol). The reaction mixture was stirred at 0° C. for 15 minutes and then at ambient temperature for 3 hours. Sodium nitrite was added to the cooled mixture which was stirred out for 15 minutes. THF was removed by rotary evaporation, the residue partitioned between EtOAc (200 ml) and D.I. water (100 ml) and the aqueous layer acidified with 1.0M HCl to pH 2. The aqueous layer was further extracted (100 ml×2 EtOAc), the combined organic layers were washed with brine (100 ml) and dried over MgSO₄. The extracts were concentrated under reduced pressure and the residue purified by column chromatography to yield the product as a colourless oil (10, 4.22 g, 24.5 mmol)

¹Hδ(CDCl₃)ppm: 1.00-1.90 (11H, m); 2.66 (1H, m); 3.78 (2H, d, J=6.0 Hz); 6.80 (1H, bs).

Step D: Preparation of Hydroxamate (12) (See Scheme 4)

A solution of the acid (10, 4.22 g, 24.5 mmol), 1-(3-Dimethylaminoprpyl)-3-ethylcarbodiimide hydrochloride (EDC, 5.64 g, 29.4 mmol) and the acetal protected hydroxylamine (11, 3.59 g, 27.0 mmol) in DMF (50 ml) was treated with 1-hydroxy-7-azabenzotriazole (HOAT, 0.05 g, 0.4 mmol) and the mixture stirred out for 16 hours at ambient temperature. The DMF was removed by rotary evaporation and the residue partitioned between EtOAc (100 ml) and 0.5M citric acid (100 ml). The organic layer was washed with D.I. water (50 ml), then brine (50 ml), dried (MgSO₄) and concentrated under reduced pressure to yield the product as a gum (12, 6.13 g, 21.3 mmol).

¹Hδ(CDCl₃)ppm: 0.93 (6H, d, J=6.7 Hz); 1.10 (2H,m); 1.45-1.90 (10H, m); 1.40 (3H, d, J=5.3 Hz); 2.31 (1H, bs); 3.31 (1H, m); 3.57 (1H, dd, J=6.6 Hz); 3.75 (2H, m); 4.98 (1H, q, J=5.3 Hz); 8.15 and 8.23 (1H total, two bs).

Step E: Preparation of Beta-Lactam (13) (See Scheme 4)

Diethyl azodicarboxylate (DEAD, 3.70 ml, 23.5 mmol) was added dropwise to a cooled (0° C.) solution of the alcohol (12, 6.13 g, 21.3 mmol) and triphenylphosphine (6.15 g, 23.5 mmol) in dry THF (80 ml) at under argon. The reaction mixture was stirred out at 0° C. for 10 minutes then for 6 hours at ambient temperature. The THF was distilled off under reduced pressure and the residue purified by column chromatography to yield the product as a yellow oil (13, 5.35 g)

¹Hδ(CDCl₃)ppm: 0.93 (6H, d, J=6.7 Hz); 1.00-1.20 (2H, m); 1.42 (3H, d, J=5.3 Hz); 1.45-2.00 (10H, m); 2.98 (1H, m); 3.27 (2H, m); 3.62 (1H, m); 3.69 (1H, m); 4.94 (1H,q, J=5.3 Hz).

Step F: Preparation of Acetal Protected Hydroxylamine (1) (See Scheme 4)

A solution of LiOH monohydrate (1.00 g, 23.8 mmol) in D.I. water (20 ml) was added portion wise to a solution of the crude β-lactam (13, 5.35g, 19.9mmol) in THF/MeOH (20 ml/60 ml) at 0° C., stirred out for 10 minutes and then allowed to reach ambient temperature over 16 hours. The solvents were removed by rotary evaporation and the residue partitioned between EtOAc (50 ml) and D.I. water (75 ml). The aqueous layer was acidified at 0° C. with 1.0M HCl to pH1, extracted with EtOAc (50+25 ml), the combined extracts washed with brine (40 ml) and dried (MgSO₄). Concentration of the extracts under reduced pressure yielded the product as a colourless oil (1, 3.39 g, 11.8 mmol).

¹Hδ(CDCl₃)ppm: 0.91 (6H, d, J=6 7 Hz); 1.00-120 (2H, m); 1.30 (3H, m); 1.35-1.69 (5H, m); 1.69-1.91 (5H, m); 2.74 (1H, m); 3.12 (2H, m); 3.25 (1H, m); 3.47 (1H, m); 4.82 (1H, m)

Step G: Preparation of Reverse Hydroxamate (2) (See Scheme 6)

A solution of the acid 1 (3.39 g, 10.8 mmol) in THF (40 ml) at 0° C. was treated with a solution of formic acetic anhydride (FAA, approx. 32 mmol) and pyridine (4.0 ml, 50 mmol) in THF at 0° C. and the mixture stirred at ambient temperature for 3 hours. THF was removed by rotary evaporation and the residue partitioned between EtOAc (75 ml) and 1.0M citric acid (75 ml). The organic layer was washed with D.I. water (2×75 ml), brine (50 ml) and dried over MgSO₄). Concentration of the extracts under reduced pressure yielded the product as a colourless oil (2, 3.69 g).

¹Hδ(CDCl₃)ppm: 0.90 (6H, dd, J=6.7 Hz); 0.96-1.21 (2H, m); 1.34 (3H, m); 1.45-1.69 (5H, m); 1.70-1.95 (5H, m); 2.80 (1H, m)*; 3.27 (1H, m); 3.48 (1H, m); 3.74 (1H, m); 3.92 (1H, m); 4.93 (1H, m)*; 8.33 (1H, m).

* denotes rotamers observed.

Step H: Preparation of N-Boc-amino amide (16)(See Scheme 5)

To a solution of the Boc amino acid (14, 435 mg, 1.5 mmol) in DCM (8 ml) at room temperature under argon was added EDC (316 mg, 1.65 mmol) and HOAT (20 mg, 0.15 mmol). The mixture was stirred at room temperature for 5 min and then 4-benzyl piperidine (15, 316 μl, 1.8 mmol) was added. After 20 h, the reaction mixture was washed with 0.5M HCl, 1M Na₂CO₃ and sat. NaCl. The organic layer was separated, dried (MgSO₄), filtered and concentrated under reduced pressure to reveal the crude product as a pale yellow oil 16. On the basis of the ESMS data, the crude material was carried forward to the next step.

m/z (ESMS, +ve ion): 470 [MH]⁺, 348, 319,176.

Step I: Preparation of Amino Amide (3) (See Scheme 5)

The N-Boc amino amide (16, 1.5 mmol) was stirred for 2 h in HCl/dioxane (1.5 ml of a sat. solution) at 20° C. ESMS indicated that the reaction was complete and the reaction mixture was concentrated under reduced pressure to yield the crude product 3 which was carried forward to the next step.

m/z (ESMS, +ve ion): 348 [MH]⁺, 176.

Step J: Preparation of Reverse Hydroxamate (4) (See Scheme 6)

To a solution of the acid (2, 236 mg, 0.75 mmol) in DMF (8 ml) at 0° C. was added O-(7-Azobenztriazol-1-yl)-N,N,N′,N′-tetramethyluronium hexafluorophosphate (HATU, 291 mg, 0.77 mmol) under argon. After 15 min. the amino amide (3, 0.75 mmol) was added followed by DIPEA (523 μl, 3.0 mmol). The yellow solution was slowly warmed to room temperature over 4 h, diluted with EtOAc (20 ml) and washed with 0.5M HCl, 1M Na₂CO₃ and sat. NaCl. The organic layer was dried (MgSO₄), filtered and concentrated under reduced pressure to yield the crude product as a pale yellow oil 4. This crude material was used in the final step.

m/z (ESMS, +ve ion): 645 [MH]⁺, 545, 348, 176.

Step K: Synthesis of N-[2-(4-Benzyl-piperidin-1-yl)-1-(3 cyano-benzyl)-2-oxo-ethyl]-2-cyclopentylmethyl-3-(formyl-hydroxy-amino)-propionamide (5). (See Scheme 6)

A solution of 4 (0.75 mmol) in 80% AcOH/H₂O (10 ml) at room temperature was stirred for 16 h after which time ESMS indicated that no starting material remained. The reaction mixture was concentrated under reduced pressure and the product was purified by preparative HPLC to give the product as a colourless oil (5, 25 mg, 6% over 4 steps).

¹Hδ(CDCl₃, rotamers): 0.11-0.19 (0.7H, m), 0.31-0.40 (0.3H, m), 0.82-1.86 (15H, m), 2.28-2.65 (4H, m), 2.80-3.12 (3H, m), 3.41-3.50 (1H, m), 3.62-4.03 (2H, m), 4.45-4.50 (1H, m), 5.08-5.16 (1H, m), 7.07-7.57 (10H, m), 7.85 (0.7H, s), 8.36 (0.3H, s)

m/z (ESMS, +ve ion): 545 [MH]⁺, 176.

HPLC (Symmetry C18, 3.5 μm, 214 nm): t_{R}=6.37 min; 92.6 area %

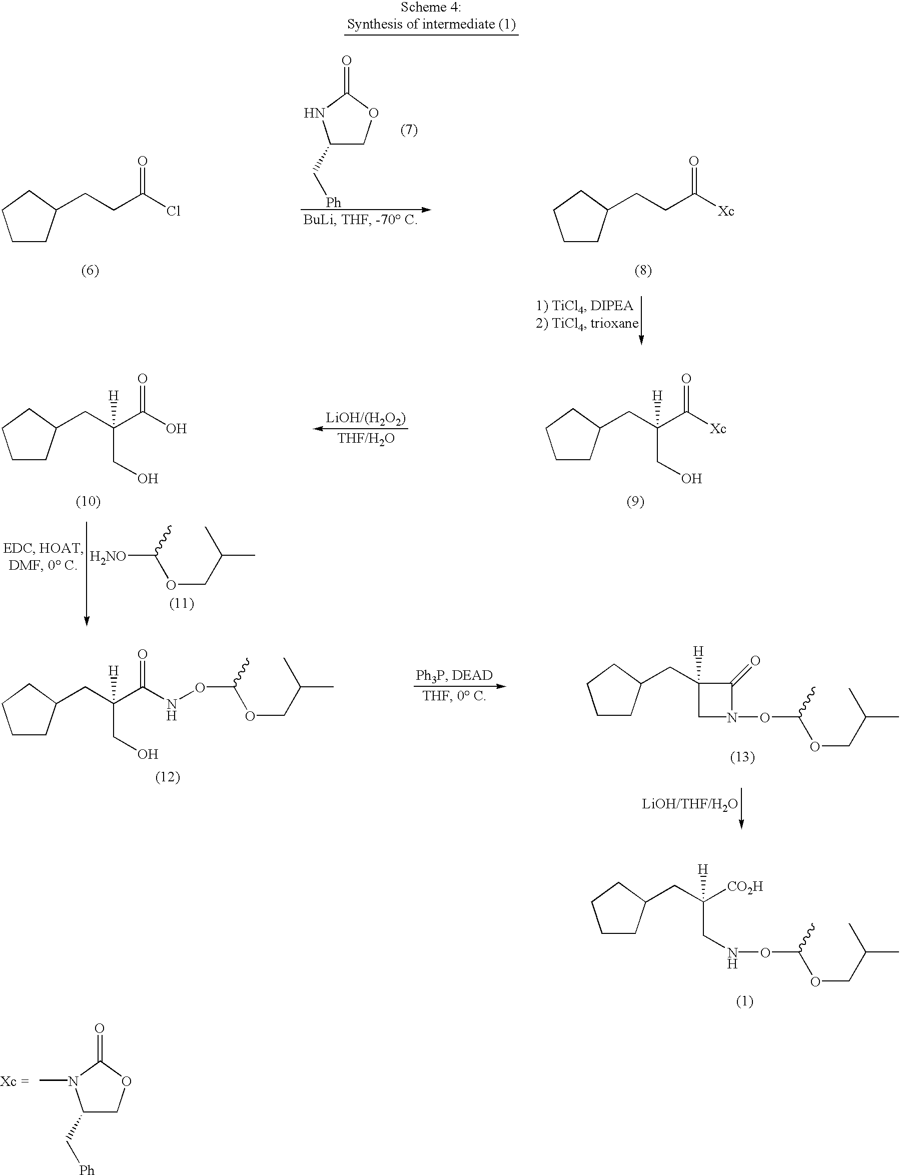

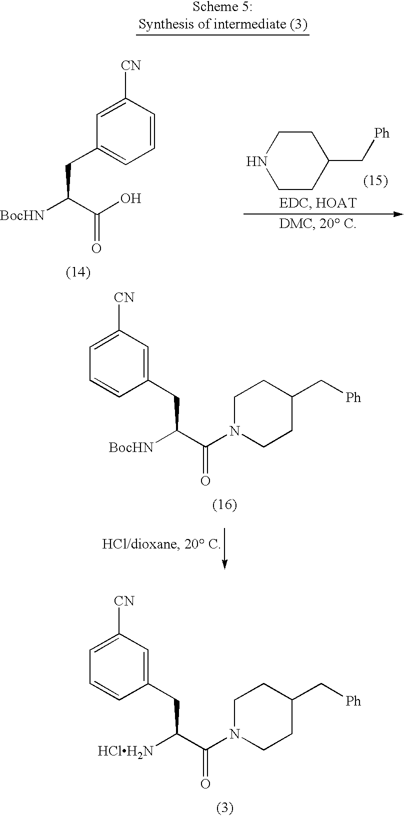

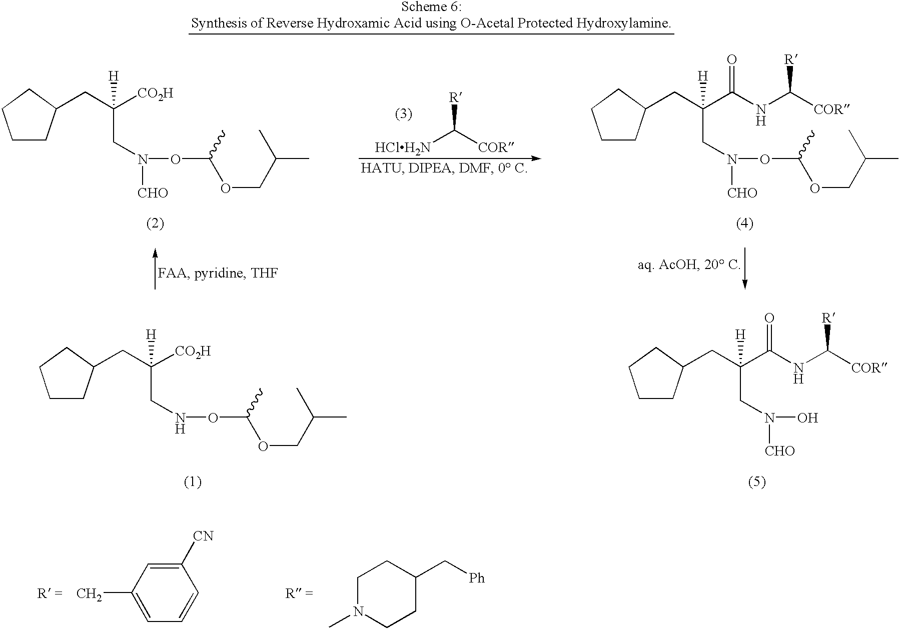

1. A compound of formula (I)
R₁CH₂CH(OR₂)ONH₂ (I)
wherein R₁ is hydrogen or methyl and R₂ is a C₁-C₆ alkyl group, or a salt thereof

2. A compound as claimed in claim 1 wherein R₁ is hydrogen.

3. A compound as claimed in claim 1 or claim 2 wherein R₂ is methyl, ethyl, n- or iso-propyl, n-, iso-, sec- or tert-butyl, n-pentyl or n-hexyl.

4. O-(1-Butoxy-ethyl)-hydroxylamine or a salt thereof.

5. O-(1-Isobutoxy-ethyl)-hydroxylamine or a salt thereof.

6. A process for converting a carboxyl group —COOH present in a precursor compound to a hydroxamic acid group —CONHOH, which process comprises condensation of the said carboxyl acid group with the amino group of a compound as claimed in any of claims 1 to 5 whereby the said carboxyl group is converted to a group of formula (II)
—CONHOCH(OR₂)(CH₂R₁) (II)
wherein R₁ and R₂ are as defined in claim 1, and subsequently the group (II) is converted to a hydroxamic acid group by acid hydrolysis.

7. A process as claimed in claim 6 wherein the carboxyl group of the precursor compound is activated for the condensation as an activated ester, an acid halide, or an acid anhydride.

8. A process as claimed in claim 6 or claim 7 wherein acid hydrolysis is effected using hydrochloric acid at ambient temperatures.

9. A process for converting a moiety of formula (IIIA), (IIIB), (IIIA¹) or (IIIB¹) present in a precursor compound 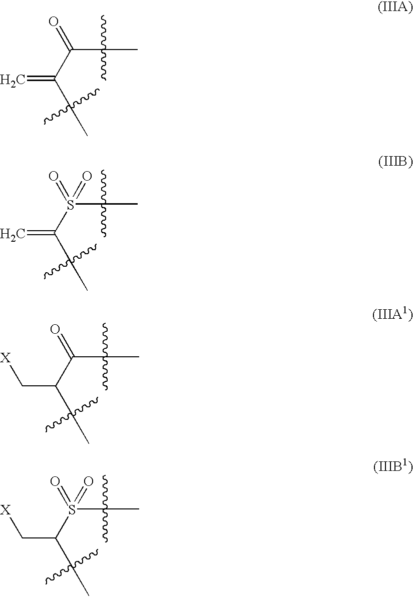
wherein X is a leaving group, to an N-formylhydroxylamino divalent moiety of formula (IVA) or (IVB) 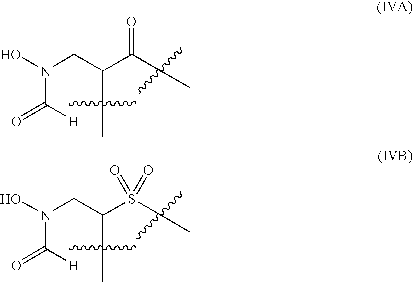
which process comprises reacting the said precursor compound with a compound of formula (I) as claimed in any of claims 1 to 5 to convert moiety (IIIA) or (IIIA¹) to a moiety (IIIC), or to convert moiety (IIIB) or (IIIB¹) to a moiety (IIID) 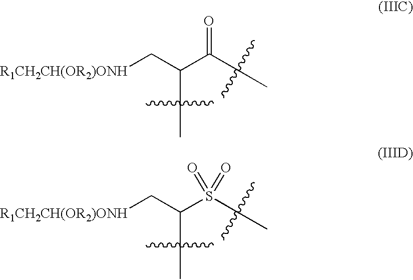
then converting moiety (IIIC) or (IIID) to moiety (IIIE) or (IIIF) by acid hydrolysis 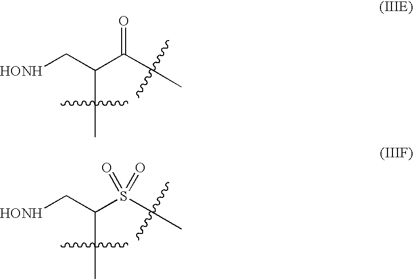
and, at the same time or subsequently, N-formylating the nitrogen of (IIIE) or (IIIF) to form moiety (IVA) or (VB).

10. A process as claimed in claim 9 wherein N-formylation of (IIIA) is effected using formic acetic anhydride, or 1-formylbenzotriazole.

11. A process for the preparation of compounds containing an N-substituted β-lactam moiety of formula (V) 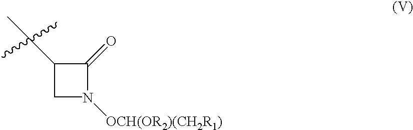
which comprises reaction of a compound containing a 1-carboxy-2-hydroxy-1-yl moiety of formula (VA) 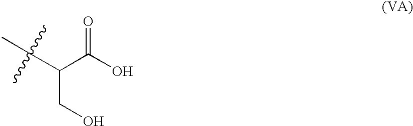
with a compound of the invention (I) thereby converting moiety (VA) to a moiety (VB) 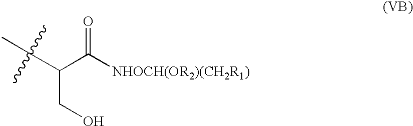
then cyclising moiety (VB) by internal amino-hydroxyl condensation to form the desired N-substituted β-lactam compound (V).

12. A process as claimed in claim 11 comprising the subsequent steps of ring opening compound (V) by acid hydrolysis to yield a compound containing a moiety of formula (VI) 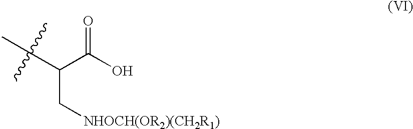
then be N-formylated compound (VI) to form a compound containing a moiety of formula (VIA) 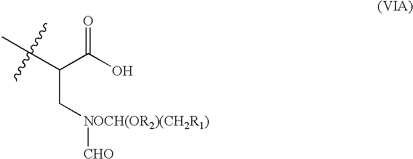
then hydrolysing compound (VA) under acid conditions to form a compound containing an N-formyl-N-hydroxylamino moiety (VB) [chemical structure]
